(19) United States
(12) Patent Application Publication
(10) Pub. No.: US 2003/0021772 A1
(43) Pub. Date: Jan. 30, 2003

(54) Method for treating effects of sleep deprivation and jet lag with NADPH and NADPH

(76) Inventor: Joerg G. D. Birkmayer, Vienna (AT)

Correspondence Address: KENYON & KENYON, ONE BROADWAY, NEW YORK, NY, 10004, US

(21) Appl. No.: 09/896,209

(22) Filed: Jun. 29, 2001

(51) Int. Cl. A61K 38/43; A61K 38/06; A61K 31/711

(52) U.S. Cl. 424/94.1; 514/47; 514/18

(57) ABSTRACT

A method for alleviating the symptoms of sleep deprivation or jet lag wherein the reduced form of nicotinamide adenine dinucleotide (NADH) or the reduced form of nicotinamide adenine dinucleotide phosphate (NADPH) or physiologically compatible salts or derivatives of NADH and/or NADPH are administered to a human being suffering from the effects. Human beings so treated exhibit an abatement of these effects, such as, for example, decreased attentiveness, decreased ability to concentrate, decreased reaction time, decreased alertness, and decreased productivity and efficiency.

### FIELD OF THE INVENTION

The present invention relates to a pharmaceutical and a method for treating the effects of sleep deprivation generally, and jet lag specifically. More particularly, the present invention relates to the use of reduced forms of nicotinamide-adenine-dinucleotide (NADH) or nicotinamide-adenine-dinucleotide phosphate (NADPH), or physiologically acceptable salts or derivatives thereof, in treating the adverse effects of sleep deprivation and jet lag.

### BACKGROUND

Every human being needs a certain amount of sleep each day in order to lead a healthy, productive life. Sleep deprivation is the condition of being deprived of this needed sleep, resulting in adverse effects on an individual, such as, for example, decreased attentiveness, decreased ability to concentrate, decreased reaction time, decreased alertness, and decreased productivity and efficiency. Sleep deprivation can be caused by, for example, sleep disorders, such as insomnia or obstructive sleep apnea, medical illnesses, shifting work schedules, depression, or flying across time zones.

Jet lag is a constellation of symptoms that occur in a human being after flying across time zones. It affects a large number of travelers and aircrew. These symptoms include: general malaise, disruption of or deprivation of sleep, gastrointestinal distress, and memory loss. In M. R. Rosekind et al., Fatigue in Operational Settings: Examples from the Aviation Environment, Hum. Factors (1994), 36(2):327-38, the authors estimate that jet lag can degrade decision-making abilities, communication and memory by 30% to 70%. The disruption of the body's entrainment of internal 24-hour cycles of temperature, sleep initiation and other activities to the day-light cycle is believed to be the trigger for jet lag. See G. Copinschi et al., Pathophysiology of Human Circadian Rhythms, Novartis Found. Symp. 2000, 227:143-57; F. W. Turek et al., Entrainment of the circadian activity rhythm to the light-dark cycle can be altered by a short-acting benzodiazepine, triazolam, J. Biol. Rhythms (1987), 2(4):249-260. Today's modern jet traveler (soldier, businessperson, athlete, or tourist) often is required to perform at a high functional level upon reaching their destination. Furthermore, the problems of jet lag have been compounded in recent years because business travelers are taking more international trips and staying fewer days at their destination.

Heretofore, research on the mitigation of jet lag has focused on methods to speed the entrainment of the circadian rhythm to the new time zone. See B. M. Stone et al., Promoting Sleep in Shiftworkers and Intercontinental Travelers, Chronobiol. Int. (1997), 14(2):133-43. These methods include sleep scheduling, phototherapy and administration of sedative and/or stimulant medications. See H. S. Koelega, Stimulant Drugs and Vigilance Performance: A Review, Psychopharmacology (1993), 111(1):1-16; K. Petrie et al., A Double-blind Trial of Melatonin as a Treatment for Jet Lag in International Cabin Crew, Biol. Psychiatry (1993), 33(7):526-30; and R. A. Wever, Use of Light to Treat Jet Lag: Differential Effects of Normal and Bright Artificial Light on Human Circadian Rhythms, Ann. N.Y. Acad. Sci. (1985), 453:282-304. Each of these methods has been found to have some merit, though each has potential adverse side effects and some are considered impractical. See J. A. Caldwell, Jr., Fatigue in the Aviation Environment: An Overview of the Causes and Effects as Well as Recommended Countermeasures, Aviat. Space Environ. Med. (1997), 68(10):932-8. Thus, a need exists for a method for efficiently treating the effects of sleep deprivation and jet lag without adverse side effects.

Nicotinamide-adenine-dinucleotide in its reduced form (“NADH”) and nicotinamide-adenine-phosphate-dinucleotide in its reduced form (“NADPH”) are physiological substances which occur in all living cells including human cells. These substances are cofactors for a variety of enzymes, the majority of which catalyze oxidation-reduction reactions. Prior to recent discoveries as to certain therapeutic properties of these compounds, their principal utility has been as diagnostic tools in clinical biochemistry and as essential components in reaction kits, for example, in measuring lactatdehydrogenase (LDH).

The most important function of NADH is its driving force for cell respiration. When using oxygen, NADH forms water and 3 ATP molecules in accordance with the following formula:

NADH+H⁺+½O₂+3Pi+3ATP→NAD⁺+3ATP+4H₂O.

Thus, with 1 NADH molecule, 3 ATP molecules are obtained which have an energy of approximately 21 kilocalories. This process is called oxidative phosphorylation. The supply of NADH and/or NADPH makes this work much easier for the organism, because it has greater energy reserves as a result.

More recently, NADH and NADPH and pharmaceutically acceptable salts thereof have been shown to be useful in the treatment of Parkinson's Disease. The effectiveness of these agents for this purpose is documented in my U.S. Pat. Nos. 4,970,200 and 5,019,561, the disclosures of which are incorporated herein by reference. In addition, I have discovered that these substances are effective in the treatment of Morbus Alzheimer (i.e., Alzheimer's Disease), which is the subject of my U.S. Pat. No. 5,444,053, and in the treatment of Chronic Fatigue Syndrome (CFS), which is the subject of my U.S. Pat. No. 5,712,259.

Prior to my recent discoveries, NADH and NADPH have never been considered for therapeutic use, probably because it was believed that these compounds are rather unstable and, hence, not capable of being absorbed by the intestines of the human body. It would have been expected that these substances would be hydrolyzed in the plasma within a few seconds.

However, studies performed recently using NADH and NADPH demonstrate that these assumptions are incorrect. When NADH and NADPH were applied intravenously to patients with Parkinson's disease, a remarkable beneficial effect was observed which lasted at least 24 hours. See U.S. Pat. Nos. 4,970,200 and 5,019,561. This indicates that NADH and NADPH are not rapidly degraded in the plasma and blood.

### SUMMARY OF THE INVENTION

It is an object of the invention to provide a new composition and method which is effective in the treatment of the effects of sleep deprivation and jet lag.

In accordance with the invention, the reduced form of nicotinamide adenine dinucleotide (NADH) or nicotinamide adenine dinucleotide phosphate (NADPH) or physiologically acceptable salts or derivatives of NADH and NADPH are administered to human beings suffering from the effects of sleep deprivation or jet lag. Daily single doses between 1 and 20 mg of NADH or NADPH, or mixtures thereof, may be used for effective treatment. Preferred doses are from 5 to 15 mg in the case of NADH and from 1 to 5 mg in the case of NADPH. It has been discovered that the administration of this endogenous substance as a pharmaceutical for the treatment of the effects of sleep deprivation or jet lag leads to surprising beneficial results without any adverse side-effects. In human beings suffering from the effects of sleep deprivation or jet lag, a clear alleviation of these effects, including but not limited to decreased attentiveness, decreased ability to concentrate, decreased reaction time, decreased alertness, and decreased productivity and efficiency, is achieved.

### BRIEF DESCRIPTION OF THE DRAWINGS

The invention will now be described in greater detail with reference to the following drawings which relate to the examples of the invention, and which are described in detail later in the specification.

FIG. 1 shows the performance accuracy by group on the ANAM Running Memory Test, a test of vigilance, across the three different test sessions (baseline refers to the testing in San Diego on the day of the flight; AM refers to testing the next day in the morning in Washington, D.C.; PM refers to testing the next day in the afternoon in Washington, D.C.). Results show a significant group by session interaction (P=0.036).

FIG. 2 shows the performance accuracy by group on the Shifting Attention Test Instruction Condition, a test of working memory, across the three different test sessions. Results show a significant group by session effect (P<0.05).

FIG. 3 shows the reaction time on the secondary task of the Complex Cognitive Assessment Battery Mark Numbers Test, a measure of divided attention, at the three different test sessions. Results show a significant group by session effect (P=0.038).

FIG. 4 shows the correct responses per minute (throughput) on the Visual Sequence Comparison Test, a measure of visual perceptual speed and accuracy, at the three different test sessions. Results show a significant group by session interaction (P=0.05).

FIG. 5 shows the percentage of subjects reporting sleepiness on the Stanford Sleepiness Scale (rating>2). There was a trend for less sleepiness in the NADH group in the PM test, with eight subjects having ratings of 1 or 2 and nine with ratings of 3 or more. In the placebo group, four subjects had ratings or 1 or 2, and 13 had ratings or 3 or more.

### DETAILED DESCRIPTION OF THE INVENTION

When NADH, NADPH, or their physiologically tolerable salts are employed in accordance with the present invention, they can be manufactured in the usual way with pharmaceutically acceptable fillers, or they can be incorporated for use into conventional galenic formulations for oral, parenteral, rectal, dermal, sublingual and nasal applications. The preparations can exist: in a solid form as tablets, capsules or coated tablets; in liquid form as a solution, suspension, spray or emulsions; in the form of suppositories, as well as in formulations having a delayed release of the active substances. Suitable nasal, sublingual, rectal and dermal delivery methods and formulations for NADH and NADPH can be found in my U.S. Pat. No. 5,750,512, which is hereby incorporated by reference.

Suitable oral forms of NADH and NADPH which can be used in the practice of the present invention are described in my U.S. Pat. No. 5,332,727, the disclosure of which is incorporated herein by reference. Both NADH and NADPH are very unstable at pHs below 7 which prevail within the confines of the human stomach. Therefore, when used in oral form, these substances must be coated with an acid-stable protective film so that they can survive the stomach environment for subsequent absorption by the intestine. Suitable acid-stable coatings are known in the art and can be applied by a conventional coating process after the active ingredients are formed into a tablet or capsule. Examples of suitable coatings are: cellulose acetate phthalate; polyvinylacetate phthalate; hydroxyl-propyl-methyl cellulose phthalate; methacryllic acid copolymers; fat-wax; shellac; zein; aqua-coating; and surerelease. Another possibility for the coating is a solution of a phthalate and a lack dry substance in isopropanol. An example of a suitable lack dry substance is sold under the name EUDRAGIT™ by Rohm Pharma. Alternatively, a protein coating in an aqueous medium may be applied. However, a sugar-coating should not be used because it will destabilize NADH.

Although NADH and/or NADPH may be used by themselves in pure form, it is preferred that they be combined in a galenic formulation with a stabilizer which is effective to inhibit oxidation of NADH and NADPH to the inactive oxidized forms NAD⁺ and NADP⁺, respectively. Most preferably, the NADH and/or NADPH is combined with both a stabilizer and a filler. It has been found that the following stabilizers are effective in inhibiting oxidation to the inactive NAD⁺ and NADP⁺ and result in the greatest shelf stability for NADH and NADPH: NaHCO₃; ascorbic acid and sodium ascorbate; tocopherols and tocopherolacetates; polyvinylpyrolidone (“PVP”) 12 (12 representing the molecular weight 12,000); PVP 25; PVP 40; PVP PF 17 (meaning polymer having a molecular weight from 17,000); PVP PF 60; methyl sulfonyl methane (“MSM”); taurine; mixture of magnesium carbonate: calcium carbonate (preferably at a weight ratio magnesium carbonate to calcium carbonate of 1:2); procaine; dihydroascorbic acid; and caffeine. Also, NADPH can be used as a stabilizer for NADH, and NADH can be used as a stabilizer for NADPH. NADH/NADPH formulations containing such stabilizers are stable for up to two years. Other various stabilizers will become apparent to those skilled in the art.

Suitable fillers for use with NADH and NADPH include: mannitol; microcrystalline cellulose; carboxymethyl cellulose; dibasic calcium phosphate; MSM; a mixture of magnesium carbonate:calcium carbonate. Other suitable fillers will become apparent to those skilled in the art. Lactose should be avoided as a filler because it reacts with NADH.

In general, a preferred formulation will include about 3 to 10% by weight NADH and/or NADPH; about 1 to 10% by weight stabilizer; and a balance of filler. Such a formulation, after being compressed into a pill or tablet and coated, is stable for over 24 months.

The NADH and/or NADPH, together with the optional stabilizer and filler, may be formed into tablets, capsules, microtablets or micropellets by processes known in the art of pill manufacturing. Tablets may be formed either by direct compression or by granulation followed by compression. Capsules may be formed by blending the components and subsequently filling capsules with the blend using conventional automatic filling equipment. Microtablets may be formed by compressing powdered or granulated components into, for example, 2 mm diameter tablets.

In the case of direct compression into tablets, a particularly preferred formulation is: NADH 5%, NaHCO₃ 10%, magnesium stearate 3%, talc 4%, silicon dioxide 1%, and mannitol 82%.

In the case of capsules, a particularly preferred formulation is: NADH 5%, NaHCO₃ 10%, polyvinylpyrolidone (PVP) 5%, microcrystalline cellulose 77%, magnesium stearate 3%, alpha-tocopherolacetate 1%, talc 3%, and silicon dioxide 1%.

Suitable physiologically acceptable salts of the coenzymes NADH and NADPH include all known physiologically acceptable acidic and basic salt-forming substances, for example: organic acids such as, for example, aliphatic or aromatic carboxylic acids, e.g., formic acid, acetic acid, succinic acid, lactic acid, malic acid, tartaric acid, citric acid, maleic acid, phenylacetic acid, benzoic acid, salicylic acid or ascorbic acid; or alkali metal hydroxides or alkaline earth metal hydroxides or salts.

For nasal administration, the NADH and/or NADPH may be taken in the form of a liquid spray or a powder spray, a gel, an ointment, an infusion, an injection or nose drops. Examples of liquid spray formulations are:

| | NADPH Liquid Spray |
|---|---|
| NADH Liquid Spray Formulation | Formulation |
| NADH 12 mg, | NADPH 2.5 mg, |
| Sodium ascorbate 36 mg, and | Sodium ascorbate 36 mg, and |
| NaHCO₃ 24 mg, | NaHCO₃ 24 mg, |
| dissolved in 1 ml deionized water | dissolved in 1 ml deionized |
| | water |
| 1 Spray dose is 0.13 ml containing | 1 Spray dose is 0.13 ml |
| 1.5 mg NADH | containing 0.32 mg NADPH |

For a powder spray, the NADH is simply ground into a fine powder and atomized from a spray bottle. Preferably, pure NADH is used for the powder spray, however, it can be used in conjunction with a filler, such as mannitol, as described below. The NADH which is inhaled through the nasal passages is absorbed by the mucosa of the nose and travels to the brain through the olfactory neural pathway. NADH administered in this manner has the same therapeutic effects as the oral form described above.

Thus, in accordance with the present invention, the NADH may be administered to the nasal cavity of a human being suffering from the effects of sleep deprivation or jet lag. The NADH (and/or NADPH) may be applied alone or in combination with other substances, for example, a pharmaceutically acceptable carrier or an agent that facilitates the transfer of the NADH through the nasal mucosa. The NADH is administered intranasally as a powder, spray, gel, ointment, infusion, injection or nose drops. The NADH is delivered to the nasal cavity. It is preferred that the NADH be delivered to the olfactory area in the upper third of the nasal cavity, and particularly to the olfactory neuroepithelium in order to promote transport of the NADH into the peripheral olfactory neurons rather than the capillaries within the respiratory epithelium. It is preferred that the transport of NADH to the brain be by means of the nervous system rather than the circulatory system so that the blood-brain barrier from the bloodstream into the brain is circumvented. However, good results can also be obtained through the bloodstream.

Surprisingly, it has been discovered that NADH (and NADPH) is capable of at least partially dissolving in the fluids that are secreted by the mucous membrane which surrounds the cilia of the olfactory receptor cells of the olfactory epithelium so that it may be absorbed into the olfactory neurons. The NADH may be combined with a carrier or other substance that fosters dissolution within nasal secretions, such as the ganglioside GM-1 or the phospolipid phosphatidylserine, or emulsifiers such as polysorbate 80. The NADH may be combined with micelles comprised of lipophilic substances which modify the permeability of the nasal membrane to enhance absorption of the NADH. Lipophilic micelles which are effective for this purpose include the gangliosides, the phospholipids and phosphatidylserine. Alternatively, the NADH may be combined with liposomes to enhance absorption of the NADH into the olfactory system.

I have also discovered that NADH (and/or NADPH) is effective in treating the effects of sleep deprivation or jet lag when administered sublingually. Like nasal administration, sublingual resorption of NADH achieves very fast results. The NADH is merely placed underneath the tongue and resorbed. Unlike the oral form of NADH described above, a sublingual form should not be coated with an acid stable protective coating.

It has also been discovered that good results are obtained when NADH (and/or NADPH) is administered rectally. However, results are not obtained as quickly as in the case of nasal or sublingual administration. NADH may be administered rectally in the form of suppositories. Suitable suppository formulations are:

| NADH Suppository Formulation | | NADPH Suppository Formulation | |
|---|---|---|---|
| NADH | 5 mg | NADPH | 2 mg |
| Sodium ascorbate | 20 mg | Sodium ascorbate | 20 mg |
| NaHCO₃ | 10 mg | NaHCO₃ | 10 mg |
| Suppository mass | 2475 mg | Suppository mass | 2478 mg |
| (Massa Novata BC, | | (Massa Novata BC, | |
| Henkel Inc) | | Henkel Inc) | |

For all forms of administration (oral, sublingual, rectal, intravenous, dermal and nasal), the NADH or NADPH, or both, may be administered alone. The NADH and/or NADPH can also be used in combination with other active ingredients such as Coenzyme Q10, L-carnitine or L-glutathion.

Specific preferred embodiments of the invention will now be described with reference to the following examples which should be regarded in an illustrative rather than a restrictive sense.

### EXAMPLES

The efficacy of a stabilized, sublingual form of reduced nicotinamide adenine dinucleotide (NADH, available commercially under the name ENADA® from Menuco Corp.) as a treatment for the effects of jet lag and sleep deprivation on human beings was examined. Healthy individuals were treated with NADH on the day following an overnight flight across North America, and the effects of NADH on their cognitive functioning were monitored. Although the sublingual form of NADH was used in these examples, any of the aforementioned forms of NADH could have been employed.

The subjects in these examples were volunteers between 35 and 55 years of age, and were in good general physical health. At a screening visit, subjects were urine tested to screen for the use of illicit drugs and pregnancy. Cognitive screening with the Trail Making Test and Symbol Digit Modalities Test (see M. D. Lezak, Neuropsychological Assessment, 3rd ed., Oxford University Press (1995)) was used to exclude subjects with cognitive function test scores>1 SD below the mean for their age. Subjects were required to be gainfully employed, to have completed 14 years of formal education, and to have none of the following conditions: history of substance abuse, obesity (body mass index>30 kg/m²), air sickness, pregnancy, nicotine use (within 6-months), mental health disorder (within 1 year), or sleep disorder. In addition, subjects were required to have a normal day/night sleep schedule in their home time zone, and to have an Epworth Sleepiness Scale rating >8 at baseline (“baseline” being the testing in San Diego on the day of the flight) (see E. Hoddes et al., Quantification of Sleepiness: A New Approach, Psychophysiology (1973), 10(4):431-6). Subjects were not permitted to be taking antidepressant medications, CNS stimulants, neuroleptics, Ginseng, Gingko Biloba, melatonin, phosphatylcholine, -acetyl carnatene, or other medications/nutritional supplements reported to enhance cognitive functioning within 90 days of the study. During the study, subjects were not permitted to use caffeine, alcohol or to take any prescription or over-the-counter medications known to enhance or depress CNS functioning.

Subjects arrived at the San Diego, Calif. test site at 1200 hours on the day of the flight. The study protocol was reviewed with the subjects and they were then each issued a laptop computer (IBM Thinkpad Model 760) and familiarized with the tests and measures to be used in the study. At approximately 1500 hours subjects were administered the entire battery of tests to establish their baseline performance (“baseline”). Subjects also received training in the method for taking the sublingual tablets. Subjects were transported to the San Diego Airport and flown to Phoenix, Ariz. where they were shuttled to a conference room at a nearby hotel, provided dinner, and readministered the battery of tests at approximately 2030 hours. Subjects were shuttled back to the airport and boarded a flight to Baltimore, Md. at 2230 hours. Thirty minutes into the flight the subjects were instructed to complete a subset of the battery of tests. Subjects were permitted to sleep after completing the tests. The duration of the flight from Phoenix to Baltimore is approximately 4 hours. Furthermore, there is a 3-hour time difference between San Diego and Baltimore. The local time in Baltimore upon arrival was approximately 0600 hours. After breakfast, subjects were shuttled to the Washington, D.C. test site where they arrived at approximately 0800 hours.

Sublingual NADH 20 mg (4 tablets of sublingual ENADAlert™ 5 mg) or an equal number of identical placebo tablets were administered by study site personnel to the subjects upon their arrival at the Washington test site. At the test site, subjects' activities were carefully monitored to avoid dehydration, exposure to daylight (subjects were kept indoors) and hunger (they were provided breakfast and lunch, which all subjects ate). Caffeine intake was strictly prohibited. Study drug was provided in moisture-proof, airtight, labeled medication bottles labeled with the subject's identification number.

Subjects completed the battery of tests 90 minutes after dosing, at approximately 0930 hours (“AM test”). Testing was repeated at 1230 hours (“PM test”), and the subjects were then dismissed from the study at 1400 hours.

The testing of the subjects consisted of computer-administered tests (including CogScreen®) to assess changes in the subjects' cognitive functioning, mood and sleepiness.

The Kay Continuous Performance Test (“KCPT”) (see R. L. Kane et Al., Computerized Assessment in Neuropsychology: A Review of Tests and Test Batteries, Neuropsychol. Rev. (1992), 3(1):1-117) was administered to provide a measure of a subject's sustained attention and vigilance. On this computer-administered cognitive test, subjects watch a computer monitor and respond only when seeing a target symbol that occurs at low frequency (i.e., 5%). The number of errors of omission (i.e., lapses of attention) and errors of commission were used to calculate total errors.

Four CogScreen sub-tests were also administered to the subjects. The Shifting Attention Test: Instruction Condition measured a subject's working memory. The subject reads a two-word instruction and then applies the instruction to the screen that follows. The accuracy, throughput (number of correct responses per minute), and median response time for correct responses were recorded. The Matching to Sample Test measured a subject's visual perceptual processing speed and working memory. The subject views a 4×4 checkerboard pattern and then on the screen that follows, the subject selects the matching checkerboard pattern. The accuracy of responses, the throughput and the median response time for correct responses were recorded. The Visual Sequence Comparison measured a subject's visual processing of number/letter sequences. The accuracy of responses, the throughput and the median response time for correct responses (VSCRTC) were recorded. The Dual Task Test: Tracking Alone measured a subject's psychomotor functioning. The subject's task is to maintain the central position of an unstable cursor that moves along a horizontal line using the left and right cursor keys. The average absolute tracking error and the number of tracking failures were recorded.

The Mark Numbers Test: Complex Cognitive Assessment Battery (“CCAB”) (see M. Samet, Complex Cognitive Assessment Battery (CCAB): Test Descriptions, Alexandria, Va., U.S. Army Research Institute (1986)) was also administered to the subjects. The CCAB is a computer-administered test measuring a subject's working memory and divided attention. The subject identifies and “marks” numbers in a spreadsheet according to an instruction (e.g., mark all even numbers between 20 and 46). While performing this task, the subject is interrupted and instructed to locate and mark the smaller or larger of two flashing numbers. After performing the secondary task the subject resumes the primary task. The total score (a derived measure of the total number of correct marks, the speed of completing the task, and performance on the secondary task) and the mean reaction time to responding to the secondary task were recorded.

Two sub-tests from the Automated Neuropsychological Assessment Metrics (“ANAM”) (see R. L. Kane et al., Computerized Assessment in Neuropsychology: A Review of Tests and Test Batteries, Neuropsychol. Rev. (1992), 3(1):1-117) battery of tests were also administered to the subjects. The first sub-test was the Running Memory Test, measuring a subject's vigilance and working memory. The subject is instructed to indicate whether or not the letter being shown on the screen is the same as the previous letter. The accuracy of responses, the throughput and the mean response time for correct responses were recorded. The second sub-test was the Math Test, measuring a subject's working memory and math reasoning. The subject is presented with 3 numbers and two operation signs (e.g., 3+5−2) and is instructed to decide whether the total is greater than 5 or less than 5. The accuracy of responses, the throughput and the mean response time for correct responses were recorded.

The subjects also self-reported their mood as part of the testing. Using the Walter Reed Mood Scale (see R. L. Kane et al., Computerized Assessment in Neuropsychology: A Review of Tests and Test Batteries, Neuropsychol. Rev. (1992), 3(1):1-117), subjects indicated their agreement or disagreement with an adjective that is presented as a description of their current mood. Subjects also indicated their levels of sleepiness using the Stanford Sleepiness Scale (see E. Hoddes et al., Quantification of Sleepiness: A New Approach, Psychophysiology (1973), 10(4):431-6), which is a 7-point self-report scale of current sleepiness, with 1 being least sleepy and 7 being most sleepy.

The statistical analysis of the testing data was accomplished as follows. For continuous measures, the effects of sublingual NADH were assessed by repeated measures analysis of variance (SPSS-PC, Version 10.7). Tests with categorical results (KCPT errors of omission and commission, Dual Task Test Hits, Stanford Sleepiness Scale) were analyzed by Chi-square test. These methods were used to provide a comparison of the NADH and placebo groups at baseline in San Diego, Calif., the morning in Washington, D.C. (AM), and the afternoon in Washington, D.C. (PM). Significant group by session interaction effects are reported. Statistical significance was set at P<0.05.

Thirty-five subjects completed the testing procedure (18 males and 17 females), with subjects being randomly assigned to the placebo and NADH groups. The two groups did not differ in age (NADH=43.9+/−6.9 years; Placebo=42.8+/−6.1 years) or gender composition (NADH=9 males/9 females; Placebo=9 males/8 females).

Although fourteen subjects reported having headaches during the study, the onset of the headache occurred before the administration of NADH or placebo for ten of these subjects. Two subjects in each group had headaches that began after the administration of either NADH or placebo. Subjects were given acetaminophen or ibuprofen for the headaches, and for eight subjects the headache resolved prior to the administration of NADH or placebo.

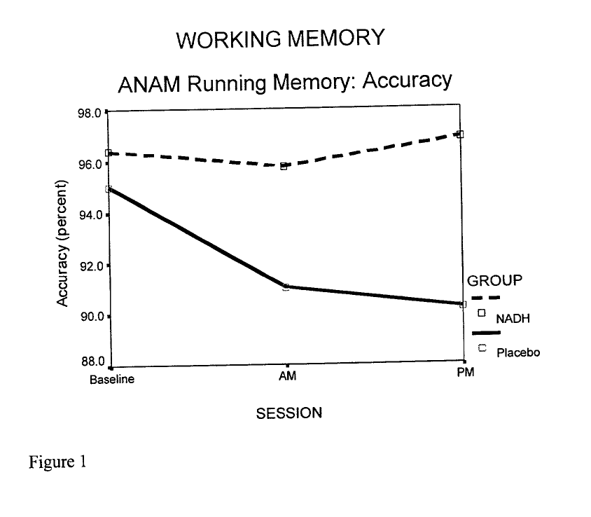

The ANAM Running Memory Test and the KCPT were the primary tests for measuring vigilance. Useable data for the ANAM Running Memory Test was obtained for only 28 subjects. Five of the subjects were not using the correct key to respond and two subjects had response times (at all 3 sessions) that were extreme outliers. For the remaining 14 NADH and 14 placebo subjects, there was a baseline difference in reaction time (P=0.005). However, the groups did not differ at baseline with respect to number of items completed or accuracy. The Group x Session interaction is significant for accuracy (P=0.036). Accuracy for placebo subjects dropped from 95% at baseline to 91% at the AM and PM testing. For NADH subjects Running Memory accuracy scores remained stable across all three sessions at approximately 96%. These results can be seen graphically in FIG. 1.

On the KCPT test, there were no group differences at baseline. Twelve of the 30 subjects (36% of NADH group, 44% of Placebo group) with a normal baseline performance (i.e., 0 to 1 omission error) made two or more errors in the AM. By the PM, 86% of NADH subjects had resumed a normal level of performance compared to only 63% of placebo subjects (P<0.08).

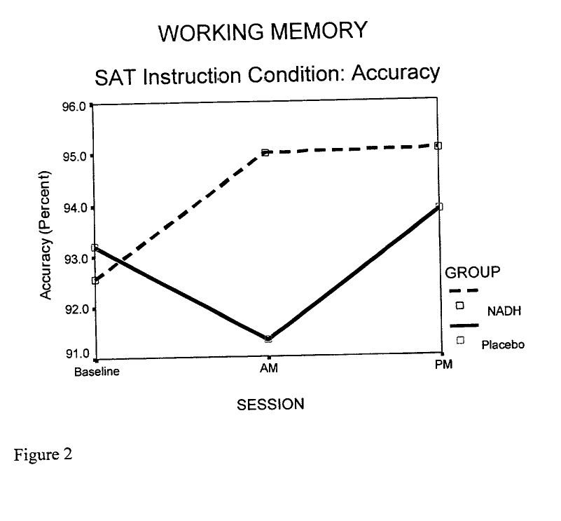

The ANAM Math Test and the Shifting Attention Test: Instruction Condition were the primary tests for measuring the working memory of the subjects. There were no baseline group differences on the Shifting Attention Test: Instruction Condition. The Group x Session effect was significant (P<0.05). Analysis of contrasts shows that subjects in the NADH group correctly completed 13.2 more problems per minute at the AM test vs. baseline, compared to 6.8 more problems correctly completed per minute for the placebo group. As can be seen in FIG. 2, for the placebo subjects accuracy dropped from 93% at baseline to 91% at the AM test, while for NADH subjects performance improved from 92.5% at baseline to 95% at the AM test session. On the ANAM Math Test, the Group x Session effect approached significance for the measure of throughput (P<0.07). For subjects in the NADH group, there was a 15% improvement relative to baseline at the AM test and an 11% improvement at the PM test. By comparison, subjects in the placebo group showed a 6% improvement at the AM test and a 4% improvement at the PM test. The mean difference between groups was not significant (P<0.08).

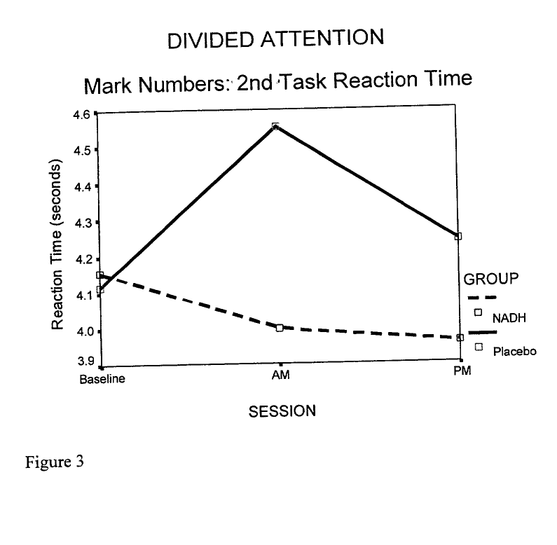

The primary measure of the subjects' divided attention was the secondary task reaction time and Total Score for the CCAB Mark Numbers Test. The Group x Session effect was significant for the secondary task reaction time (P=0.038) and for the Total Score (P=0.032). As can be seen in FIG. 3, from baseline to AM, the secondary task reaction time decreased for NADH subjects by 0.15 seconds and increased by 0.44 seconds for the placebo subjects (P=0.016). The PM test Total Score for NADH subjects increased by 77.5 points, compared to an increase of 19.2 points for placebo subjects (P=0.011).

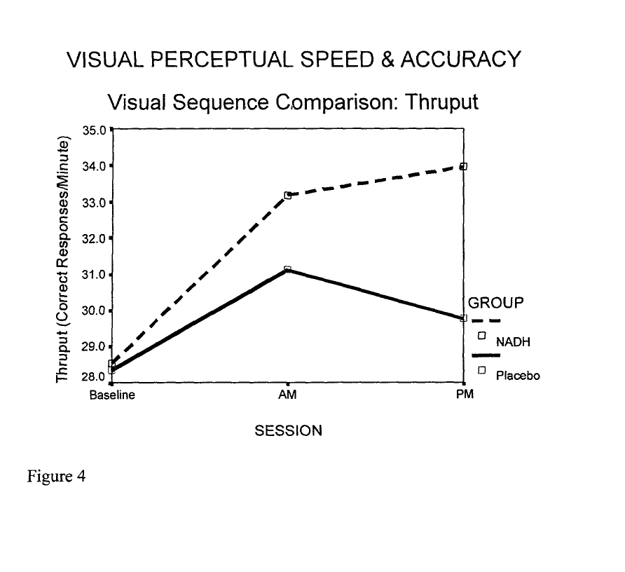

The CogScreen Matching to Sample and Visual Sequence Comparison tests provided measures of the subjects' visual perceptual speed and accuracy. For the Visual Sequence Comparison Test there was a significant Group x Session interaction for the throughput measure (correct responses per minute; P=0.05). NADH subjects correctly completed 5.4 more items per minute at the PM test compared to baseline. By comparison, the placebo subjects correctly completed 1.4 more items per minute (P=0.026). There was no significant Group x Session effect for the Matching to Sample Test. Nevertheless, as is shown in FIG. 4, the NADH group showed a tendency (P=0.078) for more improvement in throughput from baseline to PM testing: 4.9 more correct responses per minute compared to 1.0 more correct response per minute for placebo subjects.

The CogScreen Dual Task Test: Tracking Alone test provides a measure of a subject's skilled motor activity. This critical instability tracking test measures the number of tracking failures during a 90 second trial. There is generally an improvement (i.e., a practice effect) on this test reflected by fewer subjects making tracking errors over trials. This pattern of performance is evident for the NADH group where 31% had tracking failures at baseline, 33% at the AM test and 11% at the PM test. In contrast, for the subjects in the placebo group, 29% had tracking failures at baseline, 41% at the AM test and 29% at the PM test. Group comparisons show a trend for better tracking performance for NADH subjects (P<0.09).

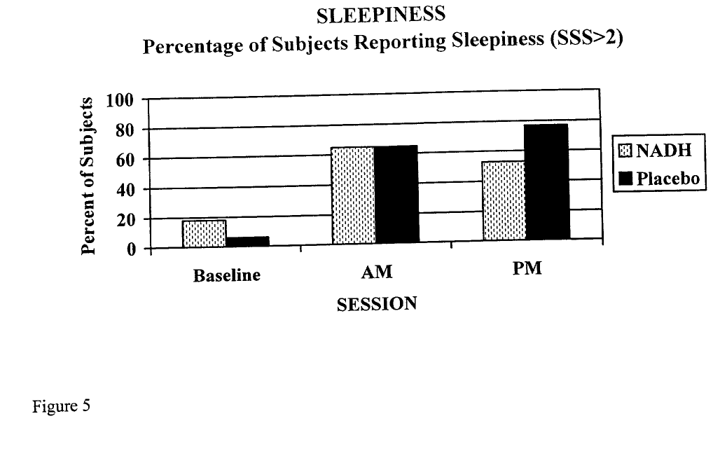

As displayed in FIG. 5, when employing the Stanford Sleepiness Scale (SSS), at baseline 14 subjects (82%) in the NADH group rated their sleepiness a 1 or 2 on the 7-point scale, and three subjects rated their sleepiness a 3. Sixteen placebo subjects (94%) rated their sleepiness a 1 or 2 at baseline, and one placebo subject had a sleepiness rating of 3. One NADH subject was an extreme outlier on the SSS and was excluded from the SSS analyses. In the AM test, both groups had identical sleepiness ratings; six in each group (35%) had a rating of 1 or 2 and eleven (65%) had ratings of 3 or more. However, in the afternoon there was a trend toward less sleepiness in the NADH group (p=0.07); eight had ratings of 1 or 2 and nine had ratings of 3 or more. For the placebo group, four subjects had ratings of 1 or 2 and thirteen had ratings of 3 or more. There were no significant differences found between groups on measures of self-reported fatigue and activity level as reported per the Walter Reed Mood Scale.

The results of these examples indicate that stabilized NADH had a beneficial effect on treating the effects of sleep deprivation and jet lag. NADH appears to mitigate the effects of jet lag on cognitive and psychomotor functions considered particularly sensitive to sedation, such as vigilance, working memory, visuomotor tracking and divided attention. In addition, NADH showed a trend to reduce the number of subjects experiencing self-reported sleepiness.

Though there were 14 subjects that reported headaches during the study, only 2 occurred after the administration of NADH. Because only 2 occurred after the treatment, we deemed that there were no adverse effects attributable to it. The absence of problems corresponds to the findings in the administration of NADH in other clinical studies (see L. M. Forsyth et Al., Therapeutic Effects of Oral NADH on the Symptoms of Patients with Chronic Fatigue Syndrome, Ann. Allergy Asthma Immunol. (1999), 82(2):185-191; J. G. Birkmayer et al., Nicotinamide Adenine Dinucleotide (NADH)—A New Therapeutic Approach to Parkinson's Disease: Comparison of Oral and Parenteral Application, Acta. Neurol. Scand. Suppl. (1993), 146:32-35; and J. G. Birkmayer, Coenzyme Nicotinamide Adenine Dinucleotide: New Therapeutic Approach for Improving Dementia of the Alzheimer Type, Ann. Clin. Lab. Sci. (1996), 26(1):1-9).

On measures of vigilance there was a notable increase in lapses of attention without NADH treatment, as reflected by omission errors on the two continuous performance tests (KCPT and ANAM Running Memory Test). These lapses of attention were most evident in the morning following the flight. By the afternoon, only 14% of NADH subjects had omission errors on the KCPT and mean accuracy on the Running Memory Test was 96%. In contrast, 37% of placebo subjects made omission errors on the KCPT and the mean accuracy on the Running Memory Test was 91%.

NADH also appears to have a protective effect on working memory, which is the ability to temporarily hold information in mind and to perform a mental operation on the information. On the morning test, subjects who received NADH showed an improvement in accuracy on the Shifting Attention Test: Instruction Condition. In sharp contrast, accuracy dropped for subjects in the placebo condition. On a second measure of working memory, the ANAM Math Test, there was also a trend for better performance with NADH treatment.

Jet lag clearly has a negative effect on divided attention, the ability to perform simultaneous mental operations. During the AM test session, subjects who received placebo were 0.44 seconds slower, compared to baseline, in their response to the secondary task on the CCAB Mark Numbers Test. By comparison, subjects who received NADH improved, compared to baseline, by 0.15 seconds on this task. Furthermore, the Total Score on the Mark Numbers Test improved significantly more for subjects who received NADH.

On two measures of visual perceptual speed and accuracy (CogScreen Visual Sequence Comparison and Matching to Sample), NADH subjects demonstrated greater improvement in the number of correct responses per minute at the afternoon test session, as compared to the placebo subjects.

The impact of the jet lag protocol on sleepiness is evident in the ratings provided by subjects on the Stanford Sleepiness Scale. During the AM test session 57.1% of the subjects in the NADH group and 62.5% of the subjects in the placebo group reported an increase in sleepiness compared to baseline. By the PM test session, 57.7% of the NADH subjects were no longer reporting an increase in sleepiness relative to their baseline rating. By comparison, only 25% of the placebo subjects were no longer reporting increased sleepiness.

Subsequently, an additional 11 subjects were tested following the same testing protocol. With the addition of these subjects (n=46), the effect on sleepiness, as measured by the Stanford Sleepiness Scale, reached significance (P<0.02). At the afternoon test, 48% of NADH subjects reported no sleepiness, compared to 18% of placebo subjects.

The public health, occupational health, and economic impact of jet lag and sleep deprivation have likely been underestimated (see M. M. Mitler et al., Catastrophes, Sleep, and Public Policy: Consensus Report, Sleep (1988), 11(1):100-9). There are an increasing number of business travelers making transcontinental and intercontinental flights. These travelers are subjected to the effects of jet lag and sleep deprivation demonstrated in the current study. The “jet lagged traveler” is more likely to experience lapses of attention (i.e., vigilance errors), to have difficulty concentrating (i.e., working memory difficulty), and to be less efficient at handling the demands of the work environment (i.e., decreased divided attention). In addition, the jet lagged traveler feels less alert, less active and more sleepy. Activities such as executive decision making or athletic performance that require attention to multiple tasks, continuous concentration and rapid interpretation of visual cues will be adversely affected by sleep deprivation and jet lag. As shown herein, stabilized NADH is effective in treating these effects of sleep deprivation and jet lag. For example, piloting an aircraft is critically dependent on vigilance, memory and visual perception, and treatments for jet lag that involve attempts to realign circadian rhythms appear to be especially impractical for commercial pilots (see A. Samel et al., Aircrew Fatigue in Long-haul Operations, Accid. Anal. Prev. (1997), 29(4):439-52). In contrast, NADH appears to be especially useful as a jet-lag or sleep-deprivation countermeasure for aircrew.

Sublingual stabilized NADH appears to be an effective treatment for the effects of jet lag and sleep deprivation on cognition and sleepiness. In the current examples, subjects receiving NADH showed less reduction of cognitive functioning and were more likely to be functioning at their baseline (pre-flight) levels than subjects who received placebo.

In the foregoing specification the invention has been described with reference to specific exemplary embodiments thereof. It will, however, be evident that various modifications and changes may be made thereto without departing from the broader spirit and scope of the invention as set forth in the appended claims. The specification should therefore be regarded in an illustrative rather than a restrictive sense.

What is claimed is:

1. A method for alleviating the effects of sleep deprivation in a human being, comprising administering to a human being exhibiting the effects of sleep deprivation an amount of NADH or NADPH or a physiologically compatible salt of NADH or NADPH which is effective to reduce or eliminate said effects of sleep deprivation.

2. The method of claim 1 wherein the NADH or NADPH or physiologically compatible salt of NADH or NADPH is administered intravenously.

3. The method of claim 1 wherein the NADH or NADPH or physiologically compatible salt of NADH or NADPH is administered orally.

4. The method of claim 1 wherein the NADH or NADPH or physiologically compatible salt of NADH or NADPH is administered sublingually.

5. The method of claim 1 wherein the NADH or NADPH or physiologically compatible salt of NADH or NADPH is administered rectally.

6. The method of claim 1 wherein the NADH or NADPH or physiologically compatible salt of NADH or NADPH is administered to a nasal passage of the human being to result in absorption of the NADH or NADPH or physiologically compatible salt of NADH or NADPH into the mucosa of the nose.

7. The method of claim 1 wherein the NADH or NADPH or physiologically compatible salt of NADH or NADPH is administered in a dose of from 1 mg to 20 mg.

8. The method of claim 1 wherein the NADH is administered in a dose of from 5 mg to 10 mg.

9. The method of claim 1 wherein the NADPH is administered in a dose of from 1 mg to 5 mg.

10. The method of claim 8 wherein said dose is administered every 24 hours.

11. The method of claim 1 wherein the NADH or NADPH or physiologically compatible salt of NADH or NADPH is administered in combination with coenzyme Q10, L-carnitine or L-glutathion.

12. A method for alleviating the effects of jet lag in a human being, comprising administering to a human being exhibiting the effects of jet lag an amount of NADH or NADPH or a physiologically compatible salt of NADH or NADPH which is effective to reduce or eliminate said effects of jet lag.

13. The method of claim 12 wherein the NADH or NADPH or physiologically compatible salt of NADH or NADPH is administered intravenously.

14. The method of claim 12 wherein the NADH or NADPH or physiologically compatible salt of NADH or NADPH is administered orally.

15. The method of claim 12 wherein the NADH or NADPH or physiologically compatible salt of NADH or NADPH is administered sublingually.

16. The method of claim 12 wherein the NADH or NADPH or physiologically compatible salt of NADH or NADPH is administered rectally.

17. The method of claim 12 wherein the NADH or NADPH or physiologically compatible salt of NADH or NADPH is administered to a nasal passage of the human being to result in absorption of the NADH or NADPH or physiologically compatible salt of NADH or NADPH into the mucosa of the nose.

18. The method of claim 12 wherein the NADH or NADPH or physiologically compatible salt of NADH or NADPH is administered in a dose of from 1 mg to 20 mg.

19. The method of claim 12 wherein the NADH is administered in a dose of from 5 mg to 10 mg.

20. The method of claim 12 wherein the NADPH is administered in a dose of from 1 mg to 5 mg.

21. The method of claim 19 wherein said dose is administered every 24 hours.

22. The method of claim 12 wherein the NADH or NADPH or physiologically compatible salt of NADH or NADPH is administered in combination with coenzyme Q10, L-carnitine or L-glutathion.

23. A method for enhancing attentiveness, alertness, concentration or reaction time in a human being, comprising administering to a human being an amount of NADH or NADPH or a physiologically compatible salt of NADH or NADPH which is effective to improve attentiveness, alertness, concentration or reaction time.

24. The method of claim 23 wherein the NADH or NADPH or physiologically compatible salt of NADH or NADPH is administered intravenously.

25. The method of claim 23 wherein the NADH or NADPH or physiologically compatible salt of NADH or NADPH is administered orally.

26. The method of claim 23 wherein the NADH or NADPH or physiologically compatible salt of NADH or NADPH is administered sublingually.

27. The method of claim 23 wherein the NADH or NADPH or physiologically compatible salt of NADH or NADPH is administered rectally.

28. The method of claim 23 wherein the NADH or NADPH or physiologically compatible salt of NADH or NADPH is administered to a nasal passage of the human being to result in absorption of the NADH or NADPH or physiologically compatible salt of NADH or NADPH into the mucosa of the nose.

29. The method of claim 23 wherein the NADH or NADPH or physiologically compatible salt of NADH or NADPH is administered in a dose of from 1 mg to 20 mg.

30. The method of claim 23 wherein the NADH is administered in a dose of from 5 mg to 10 mg.

31. The method of claim 23 wherein the NADPH is administered in a dose of from 1 mg to 5 mg.

32. The method of claim 30 wherein said dose is administered every 24 hours.

33. The method of claim 23 wherein the NADH or NADPH or physiologically compatible salt of NADH or NADPH is administered in combination with coenzyme Q10, L-carnitine or L-glutathion.
